(19) United States
(12) Patent Application Publication
(10) Pub. No.: US 2014/0170213 A1
(43) Pub. Date: Jun. 19, 2014

(54) CAPSULE FORMULATION COMPRISING MONTELUKAST AND LEVOCETIRIZINE

(71) Applicants: Yong II Kim, Suwon-si (KR); Dong Ho Kim, Seoul (KR); Taek Kwan Kwon, Suwon-si (KR); Kyeong Soo Kim, Suwon-si (KR); Jae Hyun Park, Suwon-si (KR); Jong Soo Woo, Suwon-si (KR)

(72) Inventors: Yong II Kim, Suwon-si (KR); Dong Ho Kim, Seoul (KR); Taek Kwan Kwon, Suwon-si (KR); Kyeong Soo Kim, Suwon-si (KR); Jae Hyun Park, Suwon-si (KR); Jong Soo Woo, Suwon-si (KR)

(73) Assignee: HANMI PHARM. CO., LTD., Hwaseong-si, Gyeonggi-do (KR)

(21) Appl. No.: 14/232,433

(22) PCT Filed: Jul. 11, 2012
(86) PCT No.: PCT/KR2012/005506

§ 371 Date: Jan. 13, 2014

(30) Foreign Application Priority Data
Jul. 15, 2011 (KR) 10-2011-0070680
Oct. 28, 2011 (KR) 10-2011-0111132

(51) Int. Cl. A61K 9/48; A61K 31/495; A61K 31/47

(52) U.S. Cl. CPC A61K 9/4808; A61K 31/47; A61K 31/495
USPC 424/451; 514/311; 514/255.04

(57) ABSTRACT

Disclosed is a capsule formulation for preventing or treating allergic rhinitis and asthma, which comprises two separate layers of: (1) a Montelukast layer comprising montelukast or a pharmaceutically acceptable salt thereof; and (2) a Levocetirizine layer comprising levocetirizine or a pharmaceutically acceptable salt thereof; and a method for the preparation thereof. The capsule formulation according to the present invention can completely separate two active ingredients, thereby minimizing the reactivity between them and improving product stability against aging effects, and thus, can optimize the therapeutic effects.

### FIELD OF THE INVENTION

The present invention relates to a capsule formulation for preventing or treating allergic rhinitis and asthma, which comprises two separate layers of: (1) a Montelukast layer comprising montelukast or a pharmaceutically acceptable salt thereof; and (2) a Levocetirizine layer comprising levocetirizine or a pharmaceutically acceptable salt thereof; and a method for the preparation thereof.

### BACKGROUND OF THE INVENTION

“Allergic rhinitis” refers to a symptomatic disorder of the nose induced by an IgE-mediated inflammation after allergen exposure of the membrane of the nose. The allergic rhinitis includes such symptoms as rhinorrhea, nasal obstruction, nasal itching, sneezing, ocular pruritis and so on.

“Asthma” refers to a disorder wherein inflammation of the airways causes bronchial mucosa to swell and muscular convulsion to occur in bronchi which restricts airflow into and out of the lungs, and hence. Asthma may cause such symptoms as shortness of breath, severe coughing, and in severe cases, status asthmaticus, which may result in even death.

Allergic rhinitis and asthma may develop separately; however, there is a study showing that approximately 60% of patients with allergic rhinitis have asthma as well and that 85˜95% of patients with asthma also suffer from allergic rhinitis, indicating high rates of complications between said two patient groups. Thus, there has been a need for developing a complex composition, which has an improved stability and efficacy for treatment of said two conditions.

Meanwhile, Montelukast is an antagonist inhibits cysteinyl leukotriene receptor (CysLT1), which is used for prevention and treatment of leukotriene-mediated diseases. Particularly, it has been reported that montelukast is effective in the treatment of allergic rhinitis, atopic dermatitis, chronic urticaria, sinusitis, nasal polyp, chronic obstructive pulmonary disease, conjunctivitis including nasal conjunctivitis, migraine, cystic fibrosis, viral bronchiolitis, and the like [see, e.g., S. E. Dahlen, Eur. J. Pharmacol., 533(1-3), 40-56(2006)]. Further, Singulair (MSD) comprising montelukast sodium is approved for treating asthma in adults and pediatric patients of 2 years plus, and currently available in the market.

Cetirizine is (2-(4-((4-chlorophenyl)phenylmethyl)-1-piperazinyl)ethoxy-acetic acid, and its levorotatory and dextrorotatory mirror image enantiomers were disclosed as “Levocetirizine” and “Dextrocetirizine”, respectively.

Levocetirizine can be obtained by degradation or asymmetric synthesis from a racemic mixture of Cetirizine, e.g., conventional methods disclosed in UK Patent No. 225321, or enzymatic biocatalytic hydrolysis disclosed in U.S. Pat. Nos. 4,800,162 and 5,057,427. Levocetirizine possesses antihistamine properties and hence is useful as an antiallergenic, an antihistamine agent, as well as an anticonvulsant and a bronchodialator.

International Publication No. WO 94/06429 discloses a method of treating seasonal and perennial allergic rhinitis using Levocetirizine. Also, Korean Patent No. 926410 discloses a pharmaceutical composition for treatment of allergic diseases, which comprises a segment comprising Cetirizine and a segment comprising pseudoephedrine, as active ingredients. However, the efficacy of said active ingredients, Cetirizine and pseudoephedrine, against allergic disease asthma has not been proven. Besides, consistent use of nasal decongestant, pseudoephedrine may exacerbate nasal congestion due to the rebound reaction, and may cause intractable drug induced rhinitis. Therefore, it is not recommended for more than 2 weeks of usage thereof.

Further, there has been a report relating to a pharmaceutical composition in a bilayer tablet form comprising montelukast sodium, which is stable in a basic condition, and levocetirizine dihydrochloride, which is stable in an acidic condition [R. T. Rathod, J. Indian Med. Assoc., 107(8), 562-564 (2009)]. In the preparation of said composition in a tablet form, it is very difficult to completely separate montelukast and levocetirizine from each other. Even in case a bilayer tablet is formed, it is impossible to mechanically separate each active ingredient completely. Moreover, a bilayer tablet machine is required in order to manufacture such tablets.

In addition, Montelukast is known to be unstable when exposed to light, heat, or moisture, and yields such degraded products as montelukast sulfoxide of Formula (I) and montelukast cis-isomer of Formula (II). According to M. M. Al Omani et al., when a commercially available Singulair chewable tablet was exposed to sunlight, the amount of montelukast sulfoxide was increased by 2.4% after 3 weeks; and when montelukast in 0.1 M hydrochloric acid solution was exposed to sodium, the amount of montelukast cis-isomer was increased by 14.6% [see M. M. Al Omani et al., J. Pharm. And Biomed., 45, 465-471 (2007)]. As shown in the report, it is not easy to prepare a stable montelukast product against aging.

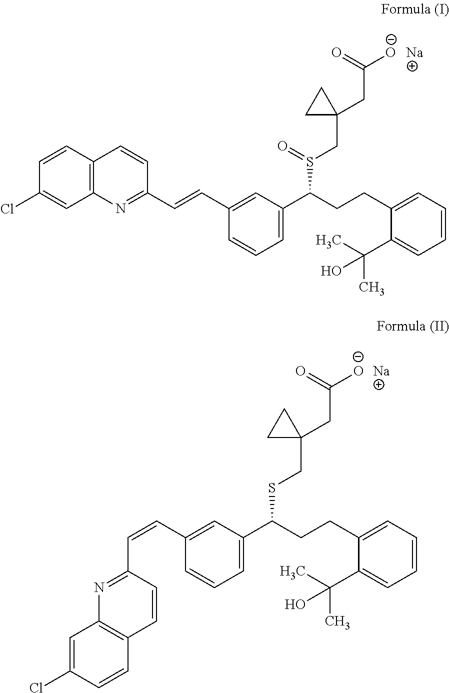 [SMILES: [Na+] ; CC(C)(O)c1ccccc1CC[C@H](c1cccc(/C=C/c2ccc3ccc(Cl)cc3n2)c1)S(=O)CC1(CC(=O)[O-])CC1 ; [Na+] ; CC(C)(O)c1ccccc1CC[C@@H](SCC1(CC(=O)[O-])CC1)c1cccc(/C=C\c2ccc3ccc(Cl)cc3n2)c1]

Levocetirizine is also instable in terms of physiochemical properties, and it is difficult to prepare a stable product against aging. There are three major degradation products of Levocetirizine, which include related compound A of Formula (III), related compound B of Formula (IV), and related compound D of Formula (V). Related compounds A and B are created via hydrolysis of Levocetirizine, and related compound D is created via ethyl ester addition of Levocetirizine. In fact, Levocetirizine shows an increased rate of formation of related compounds A, B, and D under accelerated stability conditions, and hence it is not easy to provide prescription stability.

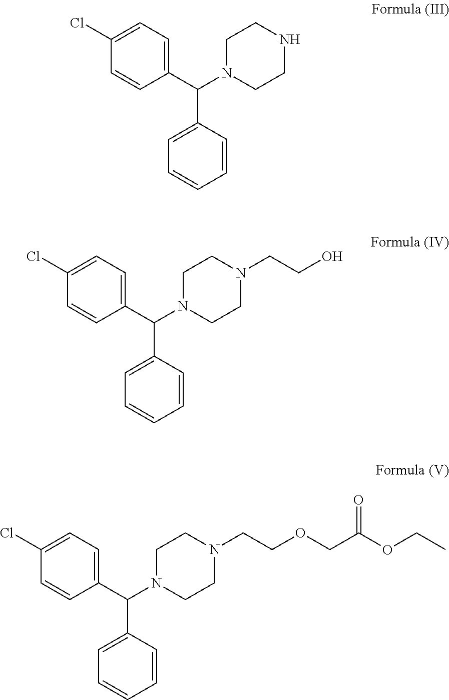 [SMILES: Clc1ccc(C(c2ccccc2)N2CCNCC2)cc1 ; OCCN1CCN(C(c2ccccc2)c2ccc(Cl)cc2)CC1 ; CCOC(=O)COCCN1CCN(C(c2ccccc2)c2ccc(Cl)cc2)CC1]

Therefore, the present inventors have developed a pharmaceutical composition comprising Montelukast as an anti-leukotriene agent with good stability while exerting no adverse effects; and Levocetirizine as an anti-histamine agent which is effective on early allergic reaction, for treatment of allergic disorders including allergic rhinitis and asthma, having stability for long-term use.

### SUMMARY OF THE INVENTION

Therefore, it is an object of the present invention to provide a pharmaceutical formulation for preventing or treating allergic rhinitis and asthma, which comprises montelukast or a pharmaceutically acceptable salt thereof; and levocetirizine or a pharmaceutically acceptable salt thereof.

It is another object of the present invention to provide a method for preparing the pharmaceutical formulation.

In accordance with the object of the present invention, there is provided a capsule formulation for preventing or treating allergic rhinitis and asthma, which comprises two separate layers of:

(1) a Montelukast layer comprising montelukast or a pharmaceutically acceptable salt thereof; and

(2) a Levocetirizine layer comprising levocetirizine or a pharmaceutically acceptable salt thereof.

In accordance with another object of the present invention, there is provided a method for preparing the capsule formulation, which comprises the steps of:

(i) mixing montelukast or a pharmaceutically acceptable salt thereof and a pharmaceutically acceptable additive, and granulating the mixture to obtain granules or forming the granules into a tablet;

(ii) mixing levocetirizine or a pharmaceutically acceptable salt thereof and a pharmaceutically acceptable additive, and granulating the mixture to obtain granules or forming the granules into a tablet; and

(iii) filling said tablet or granules of montelukast prepared in step (i) and said tablet or granules of levocetirizine prepared in step (ii) into a hard capsule to form separate layers in the capsule.

### BRIEF DESCRIPTION OF THE DRAWING

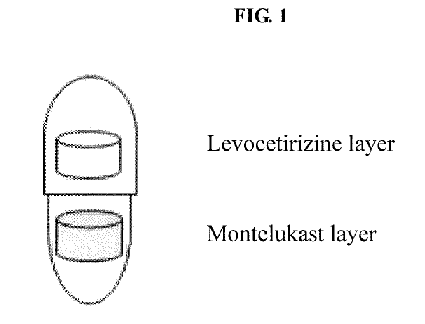

The above and other objects and features of the present invention will become apparent from the following description of the invention, when taken in conjunction with accompanying FIG. 1 which shows a schematic view of the capsule formulation of the present invention.

### DETAILED DESCRIPTION OF THE INVENTION

Hereinafter, the present invention is explained in detail.

The present invention provides a capsule formulation for preventing or treating allergic rhinitis and asthma, which comprises two separate layers of: (1) a Montelukast layer comprising montelukast or a pharmaceutically acceptable salt thereof; and (2) a Levocetirizine layer comprising levocetirizine or a pharmaceutically acceptable salt thereof.

In the present invention, the Montelukast layer and Levocetirizine layer may independently be in the form of granules or a tablet. Specifically, the capsule formulation of the present invention is a capsule formulation prepared by filling (1) a tablet or granules of montelukast comprising montelukast or a pharmaceutically acceptable salt thereof; and (2) a tablet or granules of levocetirizine comprising levocetirizine or a pharmaceutically acceptable salt thereof into a hard capsule to form two separate layers in the capsule. Wherein, at least one of the Montelukast layer and Levocetirizine layer may be in the form of a tablet.

In order to prevent any undesired side reactions caused by water, said Montelukast layer and Levocetirizine layer may be prepared without employing water or an organic solvent, or prepared in a condition which contains substantially no water or organic solvent. The amount of water content in the Montelukast layer and Levocetirizine layer is 5% or less, respectively.

The capsule formulation of the present invention employs an antihistamine agent levocetirizine as a first active ingredient to reduce early allergic reaction, as well as an anti-leukotriene agent montelukast as a second active ingredient to treat and prevent one of the major symptoms of late allergic rhinitis, i.e. nasal obstruction and asthma.

Montelukast or the pharmaceutically acceptable salt thereof used as a first active ingredient in the present invention is preferably montelukast sodium. The daily dosage amount of montelukast or the pharmaceutically acceptable salt thereof is 0.4 to 100 mg, preferably 1 to 50 mg, more preferably 2.5 to 20 mg per unit dosage form.

Levocetirizine or the pharmaceutically acceptable salt thereof used as a second active ingredient in the present invention is, for example, disclosed in European Patent Application. Nos. 0058146, 0601028 and 0801064, UK Patent Nos. 2225320 and 2225321, U.S. Pat. No. 5,478,941, and International Patent Publication No. WO 97/37982. The pharmaceutically acceptable salt of levocetirizine may include, but not limited to, an acid-addition salt of the pharmaceutically acceptable non-toxic organic or inorganic acid, such as salts of acetic acid, citric acid, maleic acid, succinic acid, ascorbic acid, hydrochloric acid, hydrobromic acid, sulfuric acid, phosphoric acid and the like; a metal salt (e.g., sodium salt or calcium salt), ammonium salt, amine salt, and amino acid salt, preferably levocetirizine dihydrochloride salt. The daily dosage amount of levocetirizine or the pharmaceutically acceptable salt thereof is 0.4 to 100 mg, preferably 1 to 50 mg, more preferably 2.5 to 20 mg per unit dosage form.

Two active ingredients according to the present invention have rapid onset time, suitable dosage amount and less harmful side effects, and thus they can be applicable to pediatric patients and show good tolerability and safety even long term use.

In the capsule formulation of the present invention, the Montelukast layer and the Levocetirizine layer, specifically, the tablet or granules forming each of said layers may comprise a pharmaceutically acceptable diluent. Suitable examples of the diluent may include microcrystalline cellulose, lactose, ludipress, mannitol, calcium phosphate monobasic, starch, low-substituted hydroxypropyl cellulose, and a mixture thereof. The diluent may be used in an amount ranging from about 1 to about 99% by weight, preferably about 5 to about 95% by weight based on the total weight of the tablet or granules.

Additionally, the tablet or granules forming each of said layers further comprise a pharmaceutically acceptable additive, e.g., a disintegrant, a binder, a stabilizing agent, a lubricant, a colorant, and the like.

Examples of the disintegrant may include any material showing a stable disintegration in a liquid environment, which is selected from the group consisting of crospovidone, sodium starch glycolate, croscarmellose sodium, low-substituted hydroxypropyl cellulose, starch, alginate or a sodium salt thereof, and a mixture thereof. Preferably, the disintegrant may be crospovidone, sodium starch glycolate, croscarmellose sodium, low-substituted hydroxypropyl cellulose or a mixture thereof. The disintegrant may be used in an amount ranging from 1 to 30% by weight, preferably 2 to 15% by weight based on the total weight of the tablet or granules.

Examples of the binder may include hydroxypropyl cellulose, hypromellose (hydroxypropyl methylcellulose), polyvinyl pyrrolidone, copovidone, macrogol, light anhydrous silicic acid, synthetic aluminum silicate, silicate derivatives such as calcium silicate or magnesium metasilicate aluminate, phosphate salts such as calcium phosphate dibasic, carbonate salts such as calcium carbonate, and a mixture thereof. The binder may be used in an amount ranging from 1 to 30% by weight, preferably 2 to 20% by weight based on the total weight of the tablet or granules.

The stabilizing agent used in the present invention may be preferably an antioxidant. The employment of the antioxidant reduces undesired side reactions caused by temperature and moisture, and thus, enhances stability against aging effects. Specific examples of the antioxidant may include butylated hydroxytoluene (BHT), butylated hydroxyanisole (BHA), ascorbic acid, ascorbyl palmitate, ethylenediaminetetraacetic acid (EDTA), sodium pyrosulfite, and a mixture thereof, preferably butylated hydroxytoluene. The stabilizing agent may be used in an amount ranging from 0.01 to 10% by weight, preferably 0.1 to 5% by weight based on the total weight of the tablet or granules.

Examples of the lubricant may include stearic acid, metal salts of stearic acid such as calcium stearate or magnesium stearate, talc, colloid silica, sugar fatty acid ester, hydrogenated vegetable oil, high melting point wax, glyceryl fatty acid ester, glycerol dibehenate and a mixture thereof. The lubricant may be used in an amount ranging from 0.3 to 5% by weight, preferably 0.5 to 3% by weight based on the total weight of the tablet or granules.

Further, each tablet comprising montelukast or levocetirizine layer may further comprise a coating layer. The coating layer may be formed on the surface of at least one selected from said tablets so as to completely separate montelukast and levocetirizine. At this time, in order to improve stabilities of montelukast and levocetirizine, the coating layer may be prepared without employing water or an organic solvent, or prepared as a water-based coating which substantially contains no water or organic solvent In the present invention, the coating substrate used for the coating layer may be conventional high molecular compounds. Examples of the coating substrate may include methylcellulose, ethylcellulose, polyvinyl alcohol, polyvinylpyrrolidone, hydroxyethylcellulose, hypromellose, but not limited thereto. The amount of the coating substrate is preferably kept at minimum so as to improve efficiency in production and provide the formulation of a size optimal for administration. Therefore, the coating substrate may be used in an amount ranging from 1 to 20% by weight, preferably 2 to 10% by weight based on the total weight of the tablet or granules.

In the capsule formulation of the present invention, the capsule may be any conventional hard capsules that are generally used in the preparation of medicine. The hard capsule substrates used in the present invention may include, e.g., gelatin, hypromellose, pullulan (NP Caps™, etc; Capsugel), or polyvinyl alcohol. In the present invention, hypromellose or pullulan having low water content is preferable to minimize degradation of active ingredients caused by water.

In the present invention, the hard capsules may have any conventional capsule size used in the preparation of medicine. The internal volume varies with size of hard capsules: No. 00 (0.95 mL), No. 0 (0.68 mL), No. 1 (0.47 mL), No. 2 (0.37 mL), No. 3 (0.27 mL) and No. 4 (0.20 mL) The size of the capsule is preferably small for patients' convenience, however, due to mass limit of the contents to be filled in the capsule, the size of the capsule used in the present invention may include No. 0, No. 1, No. 2, No. 3, and No. 4, preferably No. 1, No. 2, and No. 3.

In one embodiment of the present invention, the capsule formulation comprises (a) a montelukast tablet comprising montelukast or a pharmaceutically acceptable salt thereof; and (b) a levocetirizine tablet comprising levocetirizine or a pharmaceutically acceptable salt thereof, wherein the tablets are filled into the hard capsule.

In another embodiment of the present invention, the capsule formulation comprises (a) montelukast granules comprising montelukast or a pharmaceutically acceptable salt thereof; and (b) a levocetirizine tablet comprising levocetirizine or a pharmaceutically acceptable salt thereof, wherein the granules and the tablet are filled into the hard capsule.

In a further embodiment of the present invention, an inventive capsule formulation comprises (a) a montelukast tablet comprising montelukast or a pharmaceutically acceptable salt thereof; and (b) levocetirizine granules comprising levocetirizine or a pharmaceutically acceptable salt thereof, wherein the tablet and the granules are filled into the hard capsule.

The capsule formulation of the present invention may be used for preventing or treating allergic rhinitis and asthma, and the allergic rhinitis may include symptoms such as rhinorrhea, nasal obstruction, nasal itching, sneezing, ocular pruritis, and the like.

Further, the present invention provides a method for preparing the capsule formulation, which comprises the steps of: (i) mixing montelukast or a pharmaceutically acceptable salt thereof and a pharmaceutically acceptable additive, and granulating the mixture to obtain granules or forming the granules into a tablet; (ii) mixing levocetirizine or a pharmaceutically acceptable salt thereof and a pharmaceutically acceptable additive, and granulating the mixture to obtain granules or forming the granules into a tablet; and (iii) filling said tablet or granules of montelukast prepared in step (i) and said tablet or granules of levocetirizine prepared in step (ii) into a hard capsule to form separate layers in the capsule.

In steps (i) and (ii), the tableting process of the granules may be performed according to the conventional tableting methods with using a tablet machine. The tablet prepared may have a suitable hardness, e.g., in the range of 1 to 30 kp of the average hardness. The average hardness may be measured before forming any film coating layer on the tablets. Also, in case a tablet is produced in step (i) or (ii), the step may further comprise a process of coating the tablet.

In step (iii), the tablet or granules of montelukast, and the tablet or granules of levocetirizine may be filled into the hard capsule to form separate layers, wherein at least one selected from the Montelukast layer and the Levocetirizine layers may be in the form of a tablet.

The capsule formulation prepared in the present invention may be administered by oral, lingual, or sublingual routes.

The capsule formulation of the present invention comprises montelukast and levocetirizine separately in the hard capsule, and thus completely separate said two active ingredients. Therefore, the reactivity between two active ingredients can be minimized and the stability of the formulation is enhanced, thus optimizing therapeutic efficacy. It is also advantageous because preexisting analytical method for the evaluation of time-dependent stability of a single formulation can be also used for the inventive formulation, instead of developing a new analytical method.

The following Examples are intended to further illustrate the present invention without limiting its scope.

### Example 1

### Preparation of Capsule Formulation I

| Montelukast Layer | Quantity |
|---|---|
| Montelukast Sodium | 10.4 mg (Montelukast, 10 mg) |
| D-Mannitol | 74.3 mg |
| Microcrystalline Cellulose | 74.3 mg |
| Light anhydrous silicic acid | 5.0 mg |
| Hydroxypropyl Cellulose | 4.0 mg |
| Sodium Starch Glycolate | 30.0 mg |
| Magnesium Stearate | 2.0 mg |

| Levocetirizine Layer | Quantity |
|---|---|
| Levocetirizine Dihydrochloride | 5.0 mg |
| Ludipress | 60.5 mg |
| Microcrystalline Cellulose | 30.0 mg |
| Croscarmellose Sodium | 3.0 mg |
| Light anhydrous silicic acid | 0.5 mg |
| Magnesium Stearate | 1.0 mg |
| Opadry White (Y-1-7000) | 3.0 mg |
| Distilled Water | (15.0 mg) |

The ingredients described in Montelukast layer were mixed, and the mixture was pressed to a tablet using a round punch having a diameter of 5.5 mm to obtain a Montelukast tablet.

Meanwhile, the above procedure was repeated except for using the ingredients described in Levocetirizine layer to obtain a Levocetirizine tablet. Then, the Levocetirizine tablet was coated with a coating solution prepared by dissolving Opadry® White (Y-1-7000, Colorcon) in distilled water. Finally, said two tablets thus obtained were filled into a No. 1 hard capsule which is mainly composed of hypromellose, to obtain a capsule formulation comprising 10 mg of Montelukast and 5 mg of Levocetirizine.

### Example 2

### Preparation of Capsule Formulation II

| Montelukast Layer | Quantity |
|---|---|
| Montelukast Sodium | 5.2 mg (Montelukast, 5 mg) |
| D-Mannitol | 37.15 mg |
| Microcrystalline Cellulose | 37.15 mg |
| Light anhydrous silicic acid | 2.5 mg |
| Hydroxypropyl Cellulose | 2.0 mg |
| Sodium Starch Glycolate | 15.0 mg |
| Magnesium Stearate | 1.0 mg |

| Levocetirizine Layer | Quantity |
|---|---|
| Levocetirizine Dihydrochloride | 5.0 mg |
| Ludipress | 60.5 mg |
| Microcrystalline Cellulose | 30.0 mg |
| Croscarmellose Sodium | 3.0 mg |
| Light anhydrous silicic acid | 0.5 mg |
| Magnesium Stearate | 1.0 mg |
| Opadry White (Y-1-7000) | 3.0 mg |
| Distilled Water | (15.0 mg) |

The procedure of Example 1 was repeated except for using the ingredients and compositions described in Montelukast layer above, to obtain a capsule formulation comprising 5 mg of Montelukast and 5 mg of Levocetirizine.

### Example 3

### Preparation of Capsule Formulation III

| Montelukast Layer | Quantity | |
|---|---|---|
| Montelukast Sodium | 5.2 | mg (Montelukast, 5 mg) |
| D-Mannitol | 37.15 | mg |
| Microcrystalline Cellulose | 37.15 | mg |
| Light anhydrous silicic acid | 2.5 | mg |
| Hydroxypropyl Cellulose | 2.0 | mg |
| Sodium Starch Glycolate | 15.0 | mg |
| Magnesium Stearate | 1.0 | mg |

| Levocetirizine Layer | Quantity |
|---|---|
| Levocetirizine Dihydrochloride | 2.5 mg |
| Ludipress | 60.5 mg |
| Microcrystalline Cellulose | 30.0 mg |
| Croscarmellose Sodium | 3.0 mg |
| Light anhydrous silicic acid | 0.5 mg |
| Magnesium Stearate | 1.0 mg |
| Opadry White (Y-1-7000) | 3.0 mg |
| Distilled Water | (15.0 mg) |

The procedure of Example 1 was repeated except for using the ingredients and compositions described in Montelukast layer above and that the actual content of levocetirizine was 2.5 mg in Levocetirizine layer, to obtain a capsule formulation comprising 5 mg of Montelukast and 2.5 mg of Levocetirizine.

### Example 4

### Preparation of Capsule Formulation IV

| Montelukast Layer | Quantity | |
|---|---|---|
| Montelukast Sodium | 4.16 | mg (Montelukast, 4 mg) |
| D-Mannitol | 29.72 | mg |
| Microcrystalline Cellulose | 29.72 | mg |
| Light anhydrous silicic acid | 2.0 | mg |
| Hydroxypropyl Cellulose | 1.6 | mg |
| Sodium Starch Glycolate | 12.0 | mg |
| Magnesium Stearate | 0.8 | mg |

| Levocetirizine Layer | Quantity |
|---|---|
| Levocetirizine Dihydrochloride | 5.0 mg |
| Ludipress | 60.5 mg |
| Microcrystalline Cellulose | 30.0 mg |
| Croscarmellose Sodium | 3.0 mg |
| Light anhydrous silicic acid | 0.5 mg |
| Magnesium Stearate | 1.0 mg |
| Opadry White (Y-1-7000) | 3.0 mg |
| Distilled Water | (15.0 mg) |

The procedure of Example 1 was repeated except for using the ingredients and compositions described in Montelukast layer above, to obtain a capsule formulation comprising 4 mg of Montelukast and 5 mg of Levocetirizine.

### Example 5

### Preparation of Capsule Formulation V

| Montelukast Layer | Quantity | |
|---|---|---|
| Montelukast Sodium | 10.4 | mg (Montelukast, 10 mg) |
| D-Mannitol | 74.3 | mg |
| Microcrystalline Cellulose | 74.3 | mg |
| Light anhydrous silicic acid | 5.0 | mg |
| Hydroxypropyl Cellulose | 4.0 | mg |
| Sodium Starch Glycolate | 30.0 | mg |
| Magnesium Stearate | 2.0 | mg |
| Hypromellose | 1.73 | mg |
| Hydroxypropyl Cellulose | 1.73 | mg |
| Titanium Dioxide | 1.5 | mg |
| Red Iron Oxide | 0.04 | mg |
| Distilled Water | (15.0 | mg) |

| Levocetirizine Layer | Quantity |
|---|---|
| Levocetirizine Dihydrochloride | 5.0 mg |
| Ludipress | 60.5 mg |
| Microcrystalline Cellulose | 30.0 mg |
| Croscarmellose Sodium | 3.0 mg |
| Light anhydrous silicic acid | 0.5 mg |
| Magnesium Stearate | 1.0 mg |
| Opadry White (Y-1-7000) | 3.0 mg |
| Distilled Water | (15.0 mg) |

The ingredients described in the Montelukast layer were mixed, and the mixture was pressed to a tablet using a round punch having a diameter of 5.5 mm to obtain a Montelukast tablet. Then, the Montelukast tablet was coated with a coating solution prepared by dissolving hypromellose, hydroxypropyl cellulose, titanium dioxide and red iron oxide in distilled water.

Meanwhile, the procedure of Example 1 was repeated to obtain a Levocetirizine tablet. Finally, said two tablets thus obtained were filled into a No. 1 hard capsule which is mainly composed of hypromellose, to obtain a capsule formulation comprising 10 mg of Montelukast and 5 mg of Levocetirizine.

### Example 6

### Preparation of Capsule Formulation VI

The procedure of Example 5 was repeated except for using a hard capsule which is mainly composed of pullulan, to obtain a capsule formulation comprising 10 mg of Montelukast and 5 mg of Levocetirizine.

### Example 7

### Preparation of Capsule Formulation VII

The procedure of Example 5 was repeated except for using a hard capsule which is mainly composed of gelatin, to obtain a capsule formulation comprising 10 mg of Montelukast and 5 mg of Levocetirizine.

### Example 8

### Preparation of Capsule Formulation VIII

| Montelukast Layer (granule) | Quantity | |
|---|---|---|
| Montelukast Sodium | 10.4 | mg (Montelukast, 10 mg) |
| D-Mannitol | 74.3 | mg |
| Microcrystalline Cellulose | 74.3 | mg |
| Hydroxypropyl Cellulose | 4.0 | mg |
| Sodium Starch Glycolate | 30.0 | mg |
| Distilled Water | (20.0 | mg) |

| Levocetirizine Layer | Quantity |
|---|---|
| Levocetirizine Dihydrochloride | 5.0 mg |
| Ludipress | 60.5 mg |
| Microcrystalline Cellulose | 30.0 mg |
| Croscarmellose Sodium | 3.0 mg |
| Light anhydrous silicic acid | 0.5 mg |
| Magnesium Stearate | 1.0 mg |
| Opadry White (Y-1-7000) | 3.0 mg |
| Distilled Water | (15.0 mg) |

In accordance with the ingredients and compositions described in the Montelukast layer above, said ingredients were mixed and kneaded with a binding solution prepared by dissolving Montelukast and hydroxypropyl cellulose in distilled water, wet granulated, sieved through a 20 mesh, and dried to obtain Montelukast granules.

Meanwhile, the procedure of Example 1 was repeated to obtain a Levocetirizine tablet. Finally, the Montelukast granules and the Levocetirizine tablet were filled into a No. 1 hard capsule which is mainly composed of hypromellose, to obtain a capsule formulation comprising 10 mg of Montelukast and 5 mg of Levocetirizine.

### Example 9

### Preparation of Capsule Formulation IX

| Montelukast Layer | Quantity | |
|---|---|---|
| Montelukast Sodium | 10.4 | mg (Montelukast, 10 mg) |
| D-Mannitol | 74.3 | mg |
| Microcrystalline Cellulose | 74.3 | mg |
| Light anhydrous silicic acid | 5.0 | mg |
| Hydroxypropyl Cellulose | 4.0 | mg |
| Sodium Starch Glycolate | 30.0 | mg |
| Magnesium Stearate | 2.0 | mg |
| Hypromellose | 1.73 | mg |
| Hydroxypropyl Cellulose | 1.73 | mg |
| Titanium Dioxide | 1.5 | mg |
| Red Iron Oxide | 0.04 | mg |
| Distilled Water | (50.0 | mg) |

| Levocetirizine Layer | Quantity |
|---|---|
| Levocetirizine Dihydrochloride | 5.0 mg |
| Ludipress | 60.5 mg |
| Microcrystalline Cellulose | 30.0 mg |
| Croscarmellose Sodium | 3.0 mg |
| Hydroxypropyl Cellulose | 4.0 mg |
| Distilled Water | (15.0 mg) |

The ingredients described in Montelukast layer were mixed, and the mixture was pressed to a tablet using a round punch having a diameter of 5.5 mm to obtain a Montelukast tablet. Then, the Montelukast tablet was coated with a coating solution prepared by dissolving hypromellose, hydroxypropyl cellulose, titanium dioxide, and red iron oxide in distilled water.

Meanwhile, in accordance with the ingredients and compositions described in the Levocetirizine layer above, said ingredients were mixed and kneaded with a binding solution prepared by dissolving levocetirizine and hydroxypropyl cellulose in distilled water, wet granulated, sieved through a 20 mesh, and dried to obtain Levocetirizine granules. Finally, the Montelukast tablet and the Levocetirizine granules were filled into a No. 1 hard capsule which is mainly composed of hypromellose, to obtain a capsule formulation comprising 10 mg of Montelukast and 5 mg of Levocetirizine.

### Comparative Example 1

### Preparation of Complex Tablet

| | Quantity | |
|---|---|---|
| Montelukast Sodium | 10.4 | mg (Montelukast, 10 mg) |
| Levocetirizine Dihydrochloride | 5.0 | mg |
| D-Mannitol | 74.3 | mg |
| Microcrystalline Cellulose | 74.3 | mg |
| Light anhydrous silicic acid | 5.0 | mg |
| Hydroxypropyl Cellulose | 4.0 | mg |
| Ethanol | (20.0 | mg) |
| Sodium Starch Glycolate | 30.0 | mg |
| Magnesium Stearate | 2.0 | mg |
| Hypromellose | 1.73 | mg |
| Hydroxypropyl Cellulose | 1.73 | mg |
| Titanium Dioxide | 1.5 | mg |
| Distilled Water | (50.0 | mg) |

In accordance with the ingredients and compositions described in the above, Montelukast sodium and Levocetirizine dihydrochloride were mixed, and the mixture was kneaded with a binding solution prepared by dissolving hydroxypropyl cellulose in ethanol, wet granulated, sieved through a 20 mesh, and dried. Subsequently, light anhydrous silicic acid and magnesium stearate were added thereto, mixed, and pressed to a tablet using a tablet machine. The resulting tablet of Montelukast and Levocetirizine was then coated with a coating solution prepared by dissolving hypromellose, hydroxypropyl cellulose, titanium dioxide and red iron oxide in distilled water, to obtain a complex tablet comprising 10 mg of Montelukast and 5 mg of Levocetirizine.

### Comparative Example 2

### Preparation of Capsule Formulation

The complex tablet prepared in Comparative Example 1 was filled into a hard capsule which is mainly composed of gelatin, to obtain a capsule formulation comprising 10 mg of Montelukast and 5 mg of Levocetirizine.

### Comparative Example 3

### Preparation of Bilayer Tablet

| | Quantity | |
|---|---|---|
| Montelukast Sodium | 10.4 | mg (Montelukast, 10 mg) |
| D-Mannitol | 74.3 | mg |
| Microcrystalline Cellulose | 74.3 | mg |
| Light anhydrous silicic acid | 5.0 | mg |
| Hydroxypropyl Cellulose | 4.0 | mg |
| Ethanol | (20.0 | mg) |
| Sodium Starch Glycolate | 30.0 | mg |
| Magnesium Stearate | 2.0 | mg |
| Levocetirizine Dihydrochloride | 5.0 | mg |
| Ludipress | 60.5 | mg |
| Microcrystalline Cellulose | 30.0 | mg |
| Croscarmellose Sodium | 3.0 | mg |
| Light anhydrous silicic acid | 0.5 | mg |
| Magnesium Stearate | 1.0 | mg |
| Hypromellose | 1.73 | mg |
| Hydroxypropyl Cellulose | 1.73 | mg |
| Titanium Dioxide | 1.5 | mg |
| Distilled Water | (50.0 | mg) |

In accordance with the ingredients and compositions described in the above, said ingredients were mixed and the mixture was then kneaded with a binding solution prepared by dissolving Montelukast and hydroxypropyl cellulose in distilled water, wet granulated, sieved through a 20 mesh, and dried. Subsequently, Light anhydrous silicic acid and magnesium stearate were added thereto, mixed, and pressed to a tablet using a tablet machine.

Separately, Levocetirizine, ludipress, microcrystalline cellulose, croscarmellose sodium, light anhydrous silicic acid and magnesium stearate were mixed, and the mixture was pressed to a tablet together with the prepared Montelukast tablet to form a bilayer tablet. The bilayer tablet was then coated with a coating solution prepared by dissolving hypromellose, hydroxypropyl cellulose, titanium dioxide, and red iron oxide in distilled water, to obtain the resulting bilayer tablet comprising 10 mg of Montelukast and 5 mg of Levocetirizine.

### Experimental Example 1

### Stability Test Under Accelerated Conditions

The capsule formulations comprising Montelukast and Levocetirizine prepared in Examples 1, 5, 6 and 7, and Comparative Examples 1 and 2 were stored under accelerated storage conditions according to the following conditions. The content changes of Montelukast and Levocetirizine, and the amounts of related substances (impurities) were measured. The results are shown in Tables 3 to 5.

<Accelerated Storage Conditions>

Storage conditions: Contained in an HDPE bottle at 40° C., 75% RH

Test duration: Initial, 1, 2, 4, and 6 months

Analysis target: Montelukast and Levocetirizine

<Analysis Conditions of Montelukast and its Related Substances>

Column: Zorbax SB-Phenyl column for HPLC (Agilent Zorbax) having a stainless pipe (inner diameter of 4.6 mm×length of 25 cm) filled with diisopropyl phenethyl silica gel (particle size: 5 μm)

Eluents: A—Water containing 0.1% Trifluoroacetic acid (TFA)
B—Acetonitrile containing 0.1% TFA

TABLE 1
Elution Conditions

| Time (min) | A (%) | B (%) |
|---|---|---|
| 0 | 60 | 40 |
| 20 | 10 | 90 |
| 30 | 10 | 90 |
| 31 | 60 | 40 |
| 35 | 60 | 40 |

Detector: UV-absorption detector (absorbance at 238 nm)

Flow rate: 1.5 mL/min

Column temperature: 25° C.

<Analysis Conditions of Levocetirizine and its Related Substances>

Column: Symmetry Shield RP18 column for HPLC (Waters) having a stainless pipe (inner diameter of 4.6 mm×length of 25 cm) filled with octadecylsilyl silica gel (particle size: 5 μm)

Eluents: A—DW:Acetonitrile:10% TFA=69:30:1 (v/v)
B—DW:Acetonitrile:10% TFA=29:70:1 (v/v)

TABLE 2
Elution Conditions

| Time (min) | A (%) | B (%) |
|---|---|---|
| 0 | 100 | 0 |
| 2 | 100 | 0 |
| 30 | 25 | 75 |
| 40 | 100 | 0 |
| 50 | 100 | 0 |

Detector: UV-absorption detector (absorbance at 230 nm)

Flow rate: 1.2 mL/min

Column temperature: 30° C.

The content changes of Montelukast and Levocetirizine are shown in Table 3. Also, the changes of Montelukast related substances, i.e., Montelukast sulfoxide and Montelukast cis-isomer, as well as the changes of Levocetirizine related substances A, B, and D are shown in Tables 4 and 5, respectively.

TABLE 3
Content Changes of Montelukast and Levocetirizine

| Component | Sample | Initial | 1 month | 2 month | 4 month | 6 month |
|---|---|---|---|---|---|---|
| Montelukast | Ex. 1 | 100.0% | 99.9% | 99.7% | 99.7% | 99.6% |
| | Ex. 5 | 100.0% | 100.0% | 99.9% | 99.6% | 99.4% |
| | Ex. 6 | 100.0% | 99.8% | 99.5% | 99.4% | 99.2% |
| | Ex. 7 | 100.0% | 99.8% | 99.7% | 99.5% | 99.3% |
| | Com. | 100.0% | 99.0% | 97.6% | 96.4% | 93.6% |
| | Ex. 1 | | | | | |
| | Com. | 100.0% | 98.5% | 96.3% | 95.4% | 92.3% |
| | Ex. 2 | | | | | |
| Levocetirizine | Ex. 1 | 100.0% | 99.5% | 99.4% | 99.3% | 99.2% |
| | Ex. 5 | 100.0% | 99.8% | 99.6% | 99.4% | 99.3% |
| | Ex. 6 | 100.0% | 99.7% | 99.6% | 99.3% | 98.9% |
| | Ex. 7 | 100.0% | 99.6% | 99.5% | 99.4% | 99.2% |
| | Com. | 100.0% | 98.4% | 95.9% | 94.4% | 91.5% |
| | Ex. 1 | | | | | |
| | Com. | 100.0% | 98.1% | 94.4% | 93.6% | 92.7% |
| | Ex. 2 | | | | | |

As shown in Table 3, the capsule formulations of Examples 1, 5, 6, and 7 resulted insignificant content decreases under the accelerated test condition after 6 months, and thus exhibited exceptionally good storage stability. On the contrary, the complex tablet of Comparative Example 1 prepared by simply mixing of Montelukast and Levocetirizine, and the capsule formulation of Comparative Example 2 prepared by charging the complex tablet of Comparative Example 1 into a hard capsule, showed approximately 5% or more reduction in contents over 6 months under the accelerated storage condition.

TABLE 4
Changes of Montelukast Related Substances

| | Initial | | | Accelated condition for 6 month | | |
|---|---|---|---|---|---|---|
| | Montelukast | Montelukast | | Montelukast | Montelukast | |
| | sulfoxide | cis-isomer | | sulfoxide | cis-isomer | |
| Sample | (%) | (%) | Total (%) | (%) | (%) | Total (%) |
| Ex. 1 | 0.01 | 0.02 | 0.03 | 0.09 | 0.03 | 0.15 |
| Ex. 5 | 0.02 | 0.01 | 0.05 | 0.07 | 0.05 | 0.14 |
| Ex. 6 | 0.01 | 0.03 | 0.04 | 0.08 | 0.09 | 0.21 |
| Ex. 7 | 0.02 | 0.04 | 0.03 | 0.35 | 0.12 | 0.65 |
| Com. | 0.04 | 0.11 | 0.20 | 2.45 | 1.35 | 4.64 |
| Ex. 1 | | | | | | |
| Com. | 0.10 | 0.07 | 0.24 | 3.11 | 1.05 | 5.47 |
| Ex. 2 | | | | | | |

TABLE 5
Changes of Levocetirizine Related Substances

| | Initial | | | | Accelated condition for 6 month | | | |
|---|---|---|---|---|---|---|---|---|
| | Rel. | Rel. | Rel. | | Rel. | Rel. | Rel. | |
| | Sub. | Sub. | Sub. | | Sub. | Sub. | Sub. | |
| Sample | A (%) | B (%) | D (%) | Total (%) | A (%) | B (%) | D (%) | Total (%) |
| Ex. 1 | 0.02 | 0.01 | 0.01 | 0.06 | 0.12 | 0.11 | 0.02 | 0.35 |
| Ex. 5 | 0.02 | 0.01 | 0.00 | 0.05 | 0.09 | 0.11 | 0.02 | 0.42 |
| Ex. 6 | 0.01 | 0.02 | 0.01 | 0.06 | 0.08 | 0.05 | 0.03 | 0.29 |
| Ex. 7 | 0.03 | 0.06 | 0.02 | 0.12 | 0.35 | 0.38 | 0.02 | 0.85 |
| Com. | 0.05 | 0.07 | 0.20 | 0.35 | 1.12 | 1.08 | 3.45 | 5.95 |
| Ex. 1 | | | | | | | | |
| Com. | 0.04 | 0.05 | 0.15 | 0.33 | 1.11 | 1.04 | 3.05 | 6.42 |
| Ex. 2 | | | | | | | | |

As shown in Tables 4 and 5, the capsule formulations of Examples 1, 5, 6, and 7 resulted insignificant increases of the related substances under the accelerated test condition after 6 months, and thus exhibited exceptionally good storage stability. On the contrary, the complex tablet of Comparative Example 1 prepared by simply mixing Montelukast and Levocetirizine, and the capsule formulation of Comparative Example 2 prepared by charging the complex tablet of Comparative Example 1 into a hard capsule, showed an increase of related substances by approximately 10-fold or more under the accelerated test condition after 6 months. Therefore, it was found that a complex formulation prepared by simply mixing Montelukast and Levocetirizin degenerates its storage stability owing to the physicochemical characteristics of the active ingredients.

While the invention has been described with respect to the above specific embodiments, it should be recognized that various modifications and changes may be made to the invention by those skilled in the art which also fall within the scope of the invention as defined by the appended claims.

What is claimed is:

1. A capsule formulation for preventing or treating allergic rhinitis and asthma, which comprises two separate layers of:
(1) a Montelukast layer comprising montelukast or a pharmaceutically acceptable salt thereof; and
(2) a Levocetirizine layer comprising levocetirizine or a pharmaceutically acceptable salt thereof.

2. The capsule formulation of claim 1, wherein said Montelukast layer or said Levocetirizine layer is in the form of granules or a tablet.

3. The capsule formulation of claim 2, wherein at least one of said Montelukast layer and said Levocetirizine layer is in the form of a tablet.

4. The capsule formulation of claim 1, wherein said Montelukast layer and said Levocetirizine layer further comprise a pharmaceutically acceptable additive selected from the group consisting of a diluent, a disintegrant, a binder, a stabilizing agent, a lubricant, a colorant, and a mixture thereof.

5. The capsule formulation of claim 1, wherein said Montelukast layer and said Levocetirizine layer are prepared without employing water or an organic solvent, or prepared in a condition which contains substantially no water or organic solvent.

6. The capsule formulation of claim 1, wherein said Montelukast layer and said Levocetirizine layer contain water in an amount of 5% or less.

7. The capsule formulation of claim 3, wherein the tablet further comprises a coating layer.

8. The capsule formulation of claim 7, wherein said coating layer is prepared without employing water or an organic solvent, or prepared as a water-based coating which contains substantially no water or organic solvent.

9. The capsule formulation of claim 7, wherein said coating layer comprises a coating substrate selected from the group consisting of methylcellulose, ethylcellulose, polyvinyl alcohol, polyvinylpyrrolidone, hydroxyethylcellulose, hypromellose, and a mixture thereof.

10. The capsule formulation of claim 9, wherein said coating substrate is in an amount ranging from 1 to 20% by weight based on the total weight of the tablet.

11. The capsule formulation of claim 1, wherein said capsule is a hard capsule.

12. The capsule formulation of claim 11, wherein said capsule is made from a material selected from the group consisting of hypromellose, pullulan, gelatin and polyvinyl alcohol.

13. The capsule formulation of claim 11, wherein said capsule is made from hypromellose or pullulan.

14. The capsule formulation of claim 1, wherein said montelukast or said pharmaceutically acceptable salt thereof is contained in an amount of 2.5 mg to 20 mg per unit dosage form.

15. The capsule formulation of claim 1, wherein said levocetirizine or said pharmaceutically acceptable salt thereof is contained in an amount of 2.5 mg to 20 mg per unit dosage form.

16. The capsule formulation of claim 1, wherein said pharmaceutically acceptable salt of montelukast is montelukast sodium.

17. The capsule formulation of claim 1, wherein said pharmaceutically acceptable salt of levocetirizine is levocetirizine dihydrochloride.

18. The capsule formulation of claim 1, wherein said allergic rhinitis comprises rhinorrhea, nasal obstruction, nasal itching, sneezing or ocular pruritis.

19. A method for preparing the capsule formulation of claim 1, which comprises the steps of:
(i) mixing montelukast or a pharmaceutically acceptable salt thereof and a pharmaceutically acceptable additive, and granulating the mixture to obtain granules or forming the granules into a tablet;
(ii) mixing levocetirizine or a pharmaceutically acceptable salt thereof and a pharmaceutically acceptable additive, and granulating the mixture to obtain granules or forming the granules into a tablet; and
(iii) filling said tablet or granules of montelukast prepared in step (i) and said tablet or granules of levocetirizine prepared in step (ii) into a hard capsule to form separate layers in the capsule.

20. The method of claim 19, which further comprises coating said tablet prepared in step (i) or (ii).

21. The method of claim 19, wherein at least one of said Montelukast layer and said Levocetirizine layer is in the form of a tablet.
